(19) United States
(12) Patent Application Publication
(10) Pub. No.: US 2026/0004903 A1
(43) Pub. Date: Jan. 1, 2026

(54) SYSTEMS AND METHODS FOR DETECTING ABNORMALITIES IN PET RADIOLOGY IMAGES

(71) Applicant: MARS, INCORPORATED, McLean, VA (US)

(72) Inventor: Michael FITZKE, Verden (Aller) (DE)

(73) Assignee: MARS, INCORPORATED, McLean, VA (US)

(21) Appl. No.: 18/880,925

(22) PCT Filed: Jul. 7, 2023
(86) PCT No.: PCT/US2023/027115

§ 371 Date: Jan. 3, 2025

Related U.S. Application Data
(60) Provisional application No. 63/358,905, filed on Jul. 7, 2022.

(51) Int. Cl. G16H 15/00; G06T 7/00

(52) U.S. Cl. CPC G16H 15/00; G06T 7/0012; G06T 2207/10116; G06T 2207/20081; G06T 2207/20084; G06T 2207/30004

(57) ABSTRACT

In one embodiment, a method comprising accessing radiographic images of an animal, wherein one or more first radiographic images of the radiographic images depict the animal from one or more views, respectively, and wherein one or more second radiographic images of the radiographic images depict one or more body parts of the animal, respectively, determining disease classifications associated with the animal based on analyzing the radiographic images by a machine learning model, generating a diagnostic report associated with the animal based on the machine learning model, wherein the diagnostic report includes the disease classifications and a natural-language textual radiology report, and sending instructions for presenting the diagnostic report to a user device.

### PRIORITY

This application claims the benefit of priority to U.S. Provisional Patent Application Ser. No. 63/358,905, filed 7 Jul. 2022, the contents of which are incorporated herein by reference in its entirety.

### TECHNICAL FIELD

This disclosure relates generally to using one or more machine learning models or tools for assessing pet or animal radiology images.

### BACKGROUND

An increasing number of veterinarians utilize image based diagnostic techniques, such as X-rays, in order to diagnose or identify health issues in animals or pets. The number of veterinary trained radiologists throughout the world, however, is less than 1,100. Accordingly, many veterinarians are unable to leverage the advantages offered by image based diagnostic techniques. Even for those veterinarians who are trained in radiology, reviewing medical images can be time consuming and cumbersome. Exacerbating these difficulties, animal or pet radiology images may be oriented incorrectly and/or have missing or incorrect laterality markers. A need therefore exists for a system which can automate the processing and interpretation of diagnostic pet images and return clinically reliable results to radiology trained or non-radiology trained veterinarians.

### SUMMARY OF PARTICULAR EMBODIMENTS

In certain non-limiting embodiments, the disclosure provides systems and methods for training and using machine learning models to process, interpret, and/or analyze radiological images of animals or pets. An image can be of any format used in the diagnosis of medical conditions, such as Digital Imaging and Communications in Medicine (“DICOM”), as well as other formats which are used to display images. In particular embodiments, radiographic images can be associated with radiology reports. Conventionally for radiological image analysis, a text-specific (e.g., natural-language processing) model and an image-specific model may be trained separately based on radiology reports and radiological images, respectively. At time of deployment, the text-specific model and image-specific model may be also deployed as separate entities that can used (in principle) separate from each other. Compared to these conventional approaches, the embodiments disclosed herein can train a joint text-image-model, which can be used to directly generate and/or validate radiology reports based on radiological images. In one embodiment, the joint text-image-model can be programmed to detect abnormalities from animal or pet radiographic images.

In one embodiment, the disclosure provides systems and methods for automated detection of abnormalities from animal or pet radiographic images. In various embodiments, the analyzing and/or abnormality detection of the captured, collected, and/or received image(s) can be performed using one or more machine learning models or tools. In some embodiments, the machine learning models can include one or more neural networks. As an example and not by way of limitation, the neural networks may be convolutional neural networks (“CNN”). In one embodiment, the abnormality detection may, for example, indicate a healthy or an abnormal tissue. In one embodiment, a tissue classified as abnormal may be further be classified, for example, as cardiovascular, pulmonary structures, mediastinal structures, pleural space, and/or extra thoracic.

In some embodiments, the disclosure provides a method for abnormality detection from radiographic images of animals or pet by one or more computing systems. The method includes: accessing a plurality of radiographic images of an animal, wherein one or more first radiographic images of the plurality of radiographic images depict the animal from one or more views, respectively, and wherein one or more second radiographic images of the plurality of radiographic images depict one or more body parts of the animal, respectively; determining one or more disease classifications associated with the animal based on analyzing the plurality of radiographic images by a machine learning model; generating, based on the machine learning model, a diagnostic report associated with the animal, wherein the diagnostic report includes the one or more disease classifications and a natural-language textual radiology report; and sending, to a user device, instructions for presenting the diagnostic report.

In one embodiment, each of the plurality of radiographic images is formatted as a Digital Imaging and Communications in Medicine (“DICOM”) image.

In one embodiment, the machine learning model is based on at least one first neural network and at least one second neural network, the at least one first neural network and the at least one second neural network being coupled with each other.

In one embodiment, generating the diagnostic report includes: accessing a plurality of reference reports; encoding the plurality of reference reports into a feature space; encoding the plurality of radiographic images into the feature space; and determining the diagnostic report based on similarity search in the feature space.

In one embodiment, one of the one or more disease classifications indicates an abnormal tissue.

In one embodiment, the method further includes: identifying the abnormal tissue as at least one of cardiovascular, pulmonary structure, mediastinal structure, pleural space, or extra thoracic.

In one embodiment, the method further includes: accessing a plurality of training radiographic images, wherein the plurality of training radiographic images are associated with a plurality of training radiology reports, respectively; and training the machine learning model based on the accessed training radiograph images and their respective training radiology reports.

In one embodiment, the method further includes: preprocessing each of plurality of training radiographic images, wherein the preprocessing includes one or more of padding, random augmentation, random flip, Gaussian blur, or normalization.

In one embodiment, the method further includes: applying long document encoding to each of the plurality of training radiology reports.

In one embodiment, the method further includes: preprocessing each of plurality of training radiology reports, wherein the preprocessing includes one or more of tokenization, padding, adding a classification token, or applying an attention mask.

In one embodiment, the machine learning model includes an image encoder, a multi-image encoder, a text decoder, and a multimodal decoder.

In one embodiment, the method further includes: generating, by the image encoder, a feature map based on the plurality of radiologic images; generating, by the multi-image encoder based on the feature map, one or more multi-image keys and values; and generating, by the multimodal decoder based on the one or more multi-image keys and values and a start of sentence token, the natural-language textual radiology report.

In one embodiment, the diagnostic report further includes one or more of the plurality of radiologic images.

In various embodiments, the disclosure provides one or more computer-readable non-transitory storage media operable when executed by one or more processors to perform one or more of the methods provided by this disclosure.

In various embodiments, the disclosure provides a system comprising: one or more processors; and one or more computer-readable non-transitory storage media coupled to one or more of the processors and comprising instructions operable when executed by one or more of the processors to cause the system to perform one or more of the methods provided by this disclosure.

The embodiments disclosed herein are only examples, and the scope of this disclosure is not limited to them. Certain non-limiting embodiments can include all, some, or none of the components, elements, features, functions, operations, or steps of the embodiments disclosed herein. Embodiments according to the invention are in particular disclosed in the attached claims directed to a method. The dependencies or references back in the attached claims are chosen for formal reasons only. However any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

FIG. 1 illustrates an example architecture of the contrastive radiology captioning model.

FIG. 2 illustrates an example method for abnormality detection of radiographic images of animals.

FIG. 3 illustrates an example computer system or device used to facilitate abnormality detection from radiographic images of animals.

### DESCRIPTION OF EXAMPLE EMBODIMENTS

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them.

As used in the specification and the appended claims, the singular forms “a,” “an” and “the” include plural referents unless the context clearly dictates otherwise.

As used herein, the terms “comprises,” “comprising,” or any other variation thereof, are intended to cover a non-exclusive inclusion, such that a process, method, article, system, or apparatus that comprises a list of elements does not include only those elements but can include other elements not expressly listed or inherent to such process, method, article, or apparatus.

As used herein, the terms “animal” or “pet” as used in accordance with the present disclosure refers to domestic animals including, but not limited to, domestic dogs, domestic cats, horses, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can further refer to wild animals, including, but not limited to bison, elk, deer, venison, duck, fowl, fish, and the like. As used herein, the “feature” of the image or slide can be determined based on one or more measurable characteristics of the image or slide. For example, a feature can be a blemish in the image, a dark spot, a tissue having a various size, shape, or a light intensity level. In the detailed description herein, references to “embodiment,” “an embodiment,” “one embodiment,” “in various embodiments,” “certain embodiments,” “some embodiments,” “other embodiments,” “certain other embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

As used herein, the term “device” refers to a computing system or mobile device. For example, the term “device” can include a smartphone, a tablet computer, or a laptop computer. In particular, the computing system can comprise functionality for determining its location, direction, or orientation, such as a GPS receiver, compass, gyroscope, or accelerometer. A client device can also include functionality for wireless communication, such as BLUETOOTH communication, near-field communication (NFC), or infrared (IR) communication or communication with wireless local area networks (WLANs) or cellular-telephone network. Such a device can also include one or more cameras, scanners, touchscreens, microphones, or speakers. Client devices can also execute software applications, such as games, web browsers, or social-networking applications. Client devices, for example, can include user equipment, smartphones, tablet computers, laptop computers, desktop computers, or smartwatches.

Example processes and embodiments can be conducted or performed by a computing system or client device through a mobile application and an associated graphical user interface (“UX” or “GUI”). In certain non-limiting embodiments, the computing system or client device can be, for example, a mobile computing system-such as a smartphone, tablet computer, or laptop computer. This mobile computing system can include functionality for determining its location, direction, or orientation, such as a GPS receiver, compass, gyroscope, or accelerometer. Such a device can also include functionality for wireless communication, such as BLUETOOTH communication, near-field communication (NFC), or infrared (IR) communication or communication with wireless local area networks (WLANs), 3G, 4G, LTE, LTE-A, 5G, Internet of Things, or cellular-telephone network. Such a device can also include one or more cameras, scanners, touchscreens, microphones, or speakers. Mobile computing systems can also execute software applications, such as games, web browsers, or social-networking applications. With social-networking applications, users can connect, communicate, and share information with other users in their social networks.

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them.

In recent years, semi-supervised multi-modal artificial-intelligence (AI) models have achieved state-of-the-art results on various downstream tasks. The embodiments disclosed herein leverage the effectiveness of these methods for disease classification and report generation in the veterinary radiology domain. Specifically, a contrastive radiology captioning model is disclosed herein. The architecture of the contrastive radiology captioning model can use contrastive and captioning loss to align x-ray images and report on both a global and local level. The architecture can align multiple x-ray images to a single report as multiple different views and body parts are used to write a diagnostic report. The experimental results show that this architecture leads to significant performance increases for several radiology findings when compared to supervised training methods that use alternative labelling approaches. Ablation studies are conducted to demonstrate the importance of each architectural design choice. The text generation capabilities of the contrastive radiology captioning model highlight the potential for radiology report generation using multi-modal large language models. The contrastive radiology captioning model can be a powerful architecture for training large, unlabeled data sets with multi-image-text pair inputs.

AI systems using supervised learning methods can be used to aid veterinary radiologists in x-ray image interpretation. These methods may rely on the manual labelling of x-ray images for disease classification, a time-consuming and resource intensive process. In recent years, semi-supervised multi-modal methods have shown great success in achieving state-of-the-art performance on various downstream tasks. These methods can reduce the need for labelled data by leveraging readily available texts as ground truth labels. Dataset size and model performance tend to be positively correlated. Thus, using semi-supervised methods can increase model performance by making it possible to train with large, unlabeled datasets. This development can hold significance for the field of radiology, as models can now be trained using the vast amount of historic reports that have been routinely generated alongside x-ray images.

Furthermore, these state-of-the-art models have demonstrated the benefit that multi-modal approaches can have on unimodal model performance. Contrastive approaches may align similar texts and images by learning a joint image-text embedding space, enabling zero-shot capabilities. Moreover, optimizing generative loss for cross modal alignment has been shown to improve the ability of models to learn fine-grained local feature representations. Thus, the embodiments disclosed herein disclose a method that leverages both contrastive and generative approaches for training radiology image-text pairs for disease classification and text generation. In certain non-limiting embodiments, the disclosure provides automated techniques for detecting abnormalities from animal or pet radiographic images. One or more radiographic images can be in Digital Imaging and Communications in Medicine (“DICOM”) format. Once received, the images can be analyzed using a trained machine learning model or tool, such as a neural network model to determine abnormalities from these radiographic images. In some embodiments, this approach can use a vision encoder and decoupled text unimodal and multimodal decoder approach.

However, a particular challenge in developing models in the radiology domain can be that single patient reports typically refer to multiple images. This may be because there are usually multiple x-ray images taken during a patients visit, e.g., different body parts and views. Recent work has highlighted the importance of including relevant images for cross modal alignment with reports. The embodiments disclosed herein show that incorporating images from prior patient visits can improve model performance by reducing the ambiguity in reports resulting from missing contextual information from images. Previous work also suggests that accounting for multi-image views using a CNN-ViT architecture can lead to performance increases on radiology multi-label classification tasks). Therefore, the method disclosed herein similarly uses a hybrid CNN-Transformer architecture as the vision encoder to facilitate multi-image embedding.

Some of the example conventional work related to the embodiments disclosed herein may include RapidRead and StudyFormer. RapidRead is a deployed AI veterinary radiology system. This system can use an ensemble of CNN models for disease classification and an expert system for assessment generation. The approach in this disclosure may be compared to models from this system. The StudyFormer model may use a single image CNN encoder model and a multi-image ViT encoder model to generate study level embeddings for a patient. The architecture of the contrastive radiology captioning model may use the StudyFormer architecture as the vision encoder with some structural changes.

The embodiments disclosed herein disclose the contrastive radiology captioning model, which can be based on a self-supervised framework for vision-language processing in the radiology domain. In certain non-limiting embodiments, the contrastive radiology captioning model can be based on one or more neural networks. As an example and not by way of limitation, the neural networks can be based on convolutional neural networks, transformer based networks, or MLP-mixer. In some embodiments, the architecture of the contrastive radiology captioning model can comprise a hybrid CNN-ViT vision encoder, a text decoder, and a multi-modal decoder.

In certain non-limiting embodiments, the contrastive radiology captioning model can be trained by jointly training at least two coupled neural networks, with at least one first network for the radiographic images and at least one second network for the radiology reports. The at least one first network for the radiographic images can be considered an image encoder whereas the at least one second network for the radiology reports can be considered a text encoder. The network can be based on any suitable architecture such as Resnet50.

In some non-limiting embodiments, the joint training of the first and second networks can be based on a plurality of pairs of radiographic images and radiology reports. The contrastive radiology captioning model can be trained to predict the correct pairings of radiographic images and radiology reports in training examples. In some embodiments, the training can comprise learning a multimodal embedding space by jointly training the first and second networks to maximize the cosine similarity of the radiographic image embeddings and radiology report embeddings of correct pairs while minimizing the cosine similarity of the embeddings of the incorrect pairings.

While in some examples a neural network can train a learned weight for every input-output pair, CNNs can convolve trainable fixed-length kernels or filters along their inputs. CNNs, in other words, can learn to recognize small, primitive features (low levels) and combine them in complex ways (high levels). In particular embodiments, CNNs can be supervised, semi-supervised, or non-supervised.

In certain non-limiting embodiments, pooling, padding, and/or striding can be used to reduce the size of a CNN's output in the dimensions that the convolution is performed, thereby reducing computational cost and/or making overtraining less likely. Striding can describe a size or number of steps with which a filter window slides, while padding can include filling in some areas of the data with zeros to buffer the data before or after striding. In one embodiment, pooling, for example, can include simplifying the information collected by a convolutional layer, or any other layer, and creating a condensed version of the information contained within the layers.

In some examples, a region-based CNN (RCNN) or a one-dimensional (1-D) CNN can be used. RCNN includes using a selective search to identify one or more regions of interest in an image and extracting CNN features from each region independently for abnormality detection. Types of RCNN employed in one or more embodiments can include Fast RCNN, Faster RCNN, or Mark RCNN. In other examples, a 1-D CNN can process fixed-length time series segments produced with sliding windows. Such 1-D CNN can run in a many-to-one configuration that utilizes pooling and striding to concatenate the output of the final CNN layer. A fully connected layer can then be used to produce a detection at one or more time steps.

In some embodiments, one or more CNN models and one or more LSTM models can be combined. The combined model can include a stack of four unstrided CNN layers, which can be followed by two LSTM layers and a softmax classifier. A softmax classifier can normalize a probability distribution that includes a number of probabilities proportional to the exponentials of the input. The input signals to the CNNs, for example, are not padded, so that even though the layers are unstrided, each CNN layer shortens the time series by several samples. The LSTM layers are unidirectional, and so the softmax classification corresponding to the final LSTM output can be used in training and evaluation, as well as in reassembling the output time series from the sliding window segments. The combined model though can operate in a many-to-one configuration.

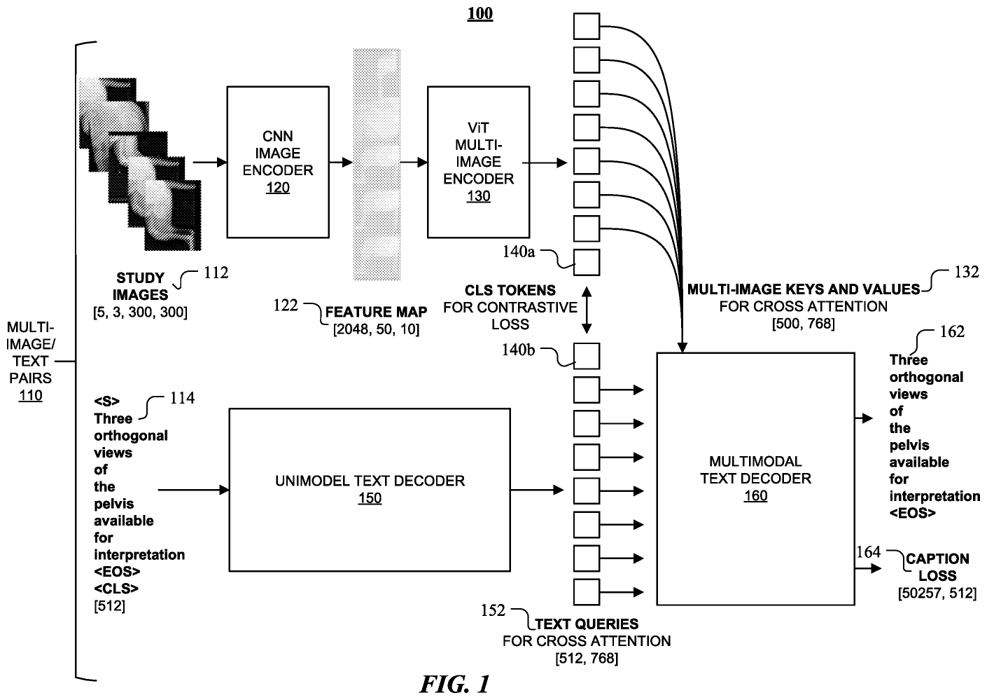

FIG. 1 illustrates an example architecture 100 of the contrastive radiology captioning model. In certain non-limiting embodiments, the contrastive radiology captioning model can be trained using a plurality of radiographic images and their associated radiology reports, i.e., multi-image/text pairs 110. The multi-image/text pairs 110 can include study images 112 and their corresponding radiology reports 114. As an example and not by way of limitation, the radio graphic images can be radiographs, CT scans, etc. The radiology reports can comprise long and unstructured textual descriptions of abnormalities compared to tags or labels. For example, a radiology report can comprise a description as “the dog has a large heart” which corresponds to a tag/label of “cardiomegaly”. As a result, training machine learning models based on radiology reports comprising long, unstructured textual descriptions can be more challenging than traditional training based on tags or labels.

In certain non-limiting embodiments, the vision encoder can be a hybrid CNN-Transformer based on the StudyFormer architecture. The CNN architecture used may be an Efficient-Net model pre-trained using multi-label classification on single view x-ray images. Each of the images in a study are first individually passed through a CNN image encoder 120, resulting in a feature map of dimensions (2048×10×10). The feature maps for all images in the study can be then concatenated to form a feature map 122 of dimensions (2048×50×10). This feature map 122 can be then passed through a ViT multi-image encoder 130, which outputs a vector representation of all images in the study with dimensions (501×768). This vector representation can include a CLS embedding 140a. In some embodiments, the ViT multi-image encoder 130 may be based on patch size of 1, depth of 12, attention heads of 12, multi-layer perceptron (MLP) dimension of 2048, and output dimensions of 500×768.

In some embodiments, both the unimodal and multi-modal text decoders can be small pre-trained generative pretrained transformer 2 (GPT2) models. The output dimensions of the unimodal text decoder 150 after embedding can be [513, 768]. This output can include a CLS embedding 140b. CLS embeddings 140 from the unimodal models can be used to calculate the contrastive loss.

The outputs from the unimodal text decoder 150 can be used as text queries 152 in the multimodal text decoder 160 for cross-attention mechanism. The outputs of the ViT multi-image encoder 130 can be used as multi-image keys and values 132. One output 162 of the multimodal text decoder 160 can include a probability distribution over the GPT2 corpus for each position in the sequence. Another output 164 of the multimodal text decoder 160 can include the caption loss, which can be calculated using the tokenized ground-truth text labels and the predicted text. The output dimensions can be [50257, 512].

In some embodiments, both GPT2 models can be trained using low-rank adaptation of large language models. The low-rank adaptation of large language models may use low rank decomposition to learn low rank matrices in the attention layers. These matrices can represent the change in the weights from the original GPT2 weights to the new task. As an example and not by way of limitation, a rank of 8 of the low-rank adaptation of large language models was used in this disclosure.

In certain non-limiting embodiments, pre-processing can be required for the radiographic images and radiology reports before training the contrastive radiology captioning model. For example, the radiographic images can be large in size, e.g., up to 456 by 456 pixels. All images can be resized to have dimensions 300×300. The maximum number of images per study may be limited to 5. In one embodiment, one can avoid the use of image cropping by padding a radiographic image. As an example and not by way of limitation, studies with less than 5 images can be padded to meet the shape requirement of [5, 3, 300, 300]. The transforms applied to the training images can include square padding, random augmentation, random flip and Gaussian blur. Normalization of [0.5, 0.5, 0.5] for both the mean and standard deviation can be applied to all images.

In some embodiments, pre-processing the radiology reports can be based on long document encoding instead of the commonly used short document encoding. In alternative embodiments, one can train a model to pre-process the radiology reports based on study notes. As an example and not by way of limitation, the radiology reports can be processed using a GPT2 tokenizer, with a maximum token length of 512. To ensure consistency, texts with less than 512 tokens can be padded with an end-of-sequence (EOS) token. Additionally, a classification token (CLS) can be added to each tokenized text to facilitate contrastive learning. An attention mask can be applied to padded tokens.

Table 1 list the definitions of symbols used in equations in the embodiments disclosed herein.

TABLE 1
Definitions of symbols used in equations

| Symbol | Definition |
|---|---|
| n | Batch size |
| F_{CE} (p, q) | The cross-entropy function applied to predictions p and targets q |
| r | Report embeddings |
| i | Image embeddings |
| r_{pred} | The predicted report embeddings rearranged such that the |
| | dimensions are [batch, vocab size, seq length] |
| rₜᵣᵤₑ | Ground-truth report embeddings |
| θ | The temperature parameter |
| L | The set of labels [0, 1, . . . , n − 1] |
| w_{c} | Contrastive loss weight |
| w_{cap} | Caption loss weight |

In certain non-limiting embodiments, contrastive loss can be used to learn discriminative features by enforcing the model to minimize the distance between similar instances and maximize the distance between dissimilar instances in the embedding space.

The caption loss can optimize the model such that it learns to generate reports that describe the input images. Given an image/and its corresponding ground-truth caption C=c₁ c₂ . . . c_{T}, where T is the length of the caption, the caption loss function can be defined as the negative log-likelihood of the correct word sequence:

$${L}_{caption} ( I , C ) = - {\sum }_{t = 1}^{T} log p ( {c}_{t} \vert I , {c}_{1 : t - 1} ) \qquad ( 1 )$$

Here, p(cₜ|I,c_{1:t=1}) represents the probability of generating the correct word cₜ at time step t, given the image/and the preceding words c_{1:t=1}.

The combined loss L_{combined} is defined as follows:

First, one can compute the similarity matrix S where each element sᵢⱼ is the dot product of ri and ij, scaled by the temperature parameter θ:

$${S}_{i , j} = 〈 {r}_{i} , {i}_{j} 〉 \cdot {e}^{θ} for i , j = 1 , 2 , … , n$$

Second, one can compute the contrastive loss L_{contrast} as the average of the cross-entropy losses computed over the similarity matrix S and its transpose S^{T}, using the labels L as targets:

$${L}_{contrast} \frac{1}{2} [ {F}_{CE} ( S , L ) + {F}_{CE} ( {S}^{T} , L ) ]$$

Third, one can compute the caption loss L_{caption} as the cross-entropy loss computed over the predicted reports r_{pred} and the true reports rₜᵣᵤₑ:

$${L}_{caption} = {F}_{CE} ( {r}_{pred} , {T}_{true} )$$

Finally, the combined loss can be the sum of the contrastive and caption loss, each scaled by their respective weights w_{c} and w_{cap}:

$${L}_{caption} = {w}_{c} \cdot {L}_{contrast} + {w}_{cap} \cdot {L}_{caption}$$

In some embodiments, the training data consisted of 3200173 image-text pairs. This was made up of 755263 studies. The validation data set consisted of 50000 image-text pairs, made up of 10446 studies. Radiology reports consist of both findings and assessments. In the embodiments disclosed herein, the model was trained on findings only.

In some embodiments, the contrastive radiology captioning model was further finetuned on a finetuning dataset. The training data of the finetuning dataset consisted of 594449 images and labels. This is made up of 145486 studies. The validation dataset consisted of 10005 images and labels, made up of 2253 studies. Each image has 41 corresponding labels that represent if a finding is present in the image or not. Each label is specific to a single x-ray view. Labels for a study were therefore determined by finding the maximum value of each label across all images in the study. Hence a pathology is considered present for a patient if the finding is present in any of the images in a study.

To validate model performance, the vision encoder was fine-tuned on a disease classification task. This was achieved by adding a classification layer with 41 outputs to the vision encoder. The model was trained using binary cross entropy loss. Several experiments were conducted using this method, including ablation studies, to understand the impact that each part of the contrastive radiology captioning model design has on classification performance.

The contrastive radiology captioning model was trained for 2 weeks on one GPU. This training lasted for 11 epochs and was stopped when the validation loss performance plateaued. Finetuning the StudyFormer vision encoder with the contrastive radiology captioning model weights took 5 days on one GPU. This was also stopped when the average precision score stopped increasing. This took 50 epochs.

In certain non-limiting embodiments, the contrastive radiology captioning model can be used to detect abnormalities in any new radiograph images. Furthermore, the contrastive radiology captioning model can generate a diagnostic report for one or more input radiographic images rather than just predict a tag or label for such images. For example, instead of predicting “cardiomegaly” for one or more radiographic images, the contrastive radiology captioning model can generate a diagnostic report comprising textual description such as “the dog has large heart” for the images. In one embodiment, to make the contrastive radiology captioning model able to generate diagnostic reports, the following steps can be used. To begin with, the contrastive radiology captioning model can encode reference diagnostic reports into a feature space. The contrastive radiology captioning model can then encode the input radiographic images into this shared feature space and perform similarity search. The contrastive radiology captioning model can further select the nearest reference diagnostic report to the input radiographic images as the outputted diagnostic report.

In certain non-limiting embodiments, besides detecting abnormalities, the contrastive radiology captioning model can determine much more detailed information regarding a detected abnormality. As an example and not by way of limitation, after detecting an abnormality, the contrastive radiology captioning model can further determine location descriptions associated with the abnormality, size descriptions associated with the abnormality, or severity descriptions associated with the abnormality.

In certain non-limiting embodiments, the contrastive radiology captioning model can be used for a variety of clinical or medical purposes. For example, a radiology image of a pet can be taken by a veterinarian or a veterinarian's assistant. That image can then be processed using the contrastive radiology captioning model. During processing, the image can be classified as normal or abnormal. If abnormal, the image can be classified as at least one of a cardiovascular, pulmonary structure, mediastinal structure, pleural space, or extra thoracic. The image can be further analyzed to determine the location descriptions, size descriptions, or severity descriptions associated with the abnormality. In some non-limiting embodiments, the image can be subclassified. For example, subclasses of pleural space can include a pleural effusion, pneumothorax, and/or pleural mass. Similarly, the image can be further analyzed to determine the location descriptions, size descriptions, or severity descriptions associated with the subclasses. The image can then be displayed to a user along with the determined abnormality class and subclass of the image. The image can be displayed on a screen or a computing device associated with the user.

In one embodiment, the contrastive radiology captioning model, and the resulting images, can be used to provide on demand second opinions for radiologists, form a basis of a service which provides veterinary hospitals with immediate assessment of radiologic images, and/or increase efficiency and productivity by allowing radiologists to focus on the pets themselves, rather than on the images.

Particular embodiments disclosed herein conducted experiments to validate the effectiveness of the contrastive radiology captioning model. The contrastive radiology captioning model was evaluated by comparing its performance to the current ensemble of models deployed in the RapidRead system. ROCAUC and average precision metrics were used to measure performance. To summarize, the contrastive radiology captioning model had higher ROCAUC for 15 findings and higher average precision for 10. Table 2 shows the results for findings that outperformed the current ensemble on at least one of the metrics.

TABLE 2
Comparison between contrastive radiology captioning model
and current ensemble ROCAUC and average precision scores.

| | Current | | Contrastive Radiology | |
|---|---|---|---|---|
| | Ensemble | | Captioning Model | |
| Finding (Disease Classification) | AUC | Precision | AUC | Precision |
| Ingesta in the stomach | 0.781 | 0.319 | 0.858 | 0.332 |
| Irregular small intestinal gas patterns | 0.774 | 0.016 | 0.952 | 0.171 |
| Irregular or granular material in the small intestines | 0.811 | 0.147 | 0.868 | 0.198 |
| Mild Small Intestinal Distention | 0.738 | 0.041 | 0.885 | 0.116 |
| Megacolon | 0.542 | 0.081 | 0.832 | 0.136 |
| Small Intestinal Obstruction | 0.955 | 0.576 | 0.976 | 0.662 |
| Large Kidney | 0.777 | 0.031 | 0.890 | 0.187 |
| Small Intestinal Plication | 0.909 | 0.163 | 0.964 | 0.182 |
| Gastric Distention | 0.973 | 0.88 | 0.984 | 0.871 |
| Mediastinal Mass Effect | 0.970 | 0.629 | 0.960 | 0.640 |
| Sternal Lymph Node Enlargement | 0.974 | 0.332 | 0.920 | 0.341 |
| Prostatic Enlargement | 0.820 | 0.554 | 0.960 | 0.530 |
| Limb Fracture | 0.905 | 0.510 | 0.960 | 0.452 |
| Rib Fracture | 0.661 | 0.049 | 0.810 | 0.025 |
| Caudal Abdominal Mass | 0.791 | 0.033 | 0.833 | 0.017 |
| Foreign Body in the Small Intestines | 0.934 | 0.471 | 0.956 | 0.341 |

To assess the impact of multi-modal training on StudyFormer performance, a StudyFormer model with Image-Net weights was also fine-tuned on the same data. The average precision and ROCAUC scores for this model were then compared to those from the contrastive radiology captioning model trained StudyFormer. This shows that there is a significant difference in performance on both metrics for the majority of findings.

In another experiment, the multimodal text decoder was removed (hence a contrastive radiology model without a captioner) to understand the impact of generative learning on the models classification performance. This means that the image encoder and text decoder weights were optimized using contrastive loss only. The results of this study showed that although the performance was better for the majority of findings when compared to the ImageNet StudyFormer, it was significantly worse than the majority of findings when compared to the contrastive radiology captioning model trained StudyFormer. The average precision results for the contrastive radiology captioning model and the ablation studies are compared in Table 3.

TABLE 3
Comparison of average precision scores across ablation studies.

| | | Contrastive | |
|---|---|---|---|
| | | Radiology | Contrastive |
| | ImageNet | Captioning | Radiology |
| Findings | StudyFormer | Model | Model |
| Aggressive Bone Lesion | 0.122 | 0.177 | 0.131 |
| Caudal Abdominal Mass | 0.090 | 0.342 | 0.015 |
| Constipation/Obstipation | 0.068 | 0.342 | 0.127 |
| Cranial Abdominal Mass | 0.307 | 0.189 | 0.120 |
| Decreased serosal detail | 0.604 | 0.805 | 0.747 |
| Degenerative Joint Disease | 0.613 | 0.787 | 0.722 |
| Esophagal Dilation | 0.568 | 0.744 | 0.668 |
| Fat Opacity Mass (e.g. lipoma) | 0.677 | 0.831 | 0.704 |
| Foreign Body in the Small Intestines | 0.188 | 0.341 | 0.336 |
| Gall Bladder Calculi | 0.062 | 0.146 | 0.071 |
| Gastric Dilatation Volvulus | 0.009 | 0.034 | 0.020 |
| Gastric Distention | 0.767 | 0.871 | 0.833 |
| Gastric Foreign Material (debris) | 0.557 | 0.699 | 0.649 |
| Hepatic Mineralization | 0.017 | 0.044 | 0.028 |
| Ingesta in the stomach | 0.190 | 0.332 | 0.143 |
| Irregular or granular material in the small intestines | 0.101 | 0.198 | 0.134 |
| Irregular small intestinal gas patterns | 0.014 | 0.171 | 0.053 |
| Large Kidney | 0.092 | 0.187 | 0.018 |
| Limb Fracture | 0.193 | 0.452 | 0.365 |
| Luxation | 0.178 | 0.310 | 0.360 |
| Mediastinal Mass Effect | 0.338 | 0.640 | 0.619 |
| Mediastinal Widening | 0.597 | 0.775 | 0.741 |
| Megacolon | 0.027 | 0.136 | 0.026 |
| Mid Abdominal Mass | 0.374 | 0.472 | 0.396 |
| Mild Small Intestinal Distention | 0.038 | 0.116 | 0.102 |
| Misshapen Kidney(s) | 0.108 | 0.371 | 0.473 |
| Pneumothorax | 1.000 | 0.599 | 0.840 |
| Prostatic Enlargement | 0.060 | 0.530 | 0.055 |
| Pulmonary Alveolar | 0.793 | 0.881 | 0.854 |
| Pulmonary Interstitial - Nodule(s) (Under 1 cm) | 0.411 | 0.625 | 0.507 |
| Pulmonary Mass (Over 1 cm) | 0.367 | 0.589 | 0.577 |
| Pulmonary Vascular | 0.610 | 0.694 | 0.656 |
| Pyloric outflow obstruction | 0.017 | 0.030 | 0.033 |
| Renal Mineralization | 0.105 | 0.448 | 0.398 |
| Rib Fracture | 0.341 | 0.025 | 0.016 |
| Sign(s) of IVDD | 0.594 | 0.709 | 0.656 |
| Sign(s) of Pleural Effusion | 0.778 | 0.922 | 0.883 |
| Small Intestinal Obstruction | 0.392 | 0.663 | 0.442 |
| Small Intestinal Plication | 0.012 | 0.182 | 0.074 |
| Small Kidney | 0.319 | 0.445 | 0.405 |
| Small Liver | 0.003 | 0.004 | 0.002 |
| Splenomegaly | 0.461 | 0.672 | 0.461 |
| Sternal Lymph Node Enlargement | 0.079 | 0.339 | 0.380 |
| Stifle Effusion | 0.735 | 0.877 | 0.793 |
| Subcutaneous Mass | 0.680 | 0.772 | 0.730 |
| Subcutaneous Nodule | 0.045 | 0.093 | 0.067 |
| Urinary Bladder Calculus/Calculi | 0.084 | 0.601 | 0.351 |
| Uterine Enlargement | 0.191 | 0.494 | 0.093 |

The text generation capabilities of contrastive radiology captioning model was tested using unseen test data. First, multi-image x-ray embeddings were generated using the StudyFormer vision encoder. These were then used in the multimodal decoder as keys and values. A start of sentence token was used as the initial query. The keys, values and queries were then used by the multi-modal decoder to autoregressively generate a text sequence, which in this case was a diagnostic report. This text was compared to human reports. The comparison demonstrates that the model can generate accurate reports that closely resemble how a human would interpret the images and write a report on them. It also shows that the model hallucinates a significant amount of information that is not present in the human text. The extent to which the generated and human text aligned varied significantly between studies.

In alternative embodiments, one may train a machine learning model configured for detecting abnormalities from animal or pet radiographic images based on a pre-trained Resnet50 architecture instead of the architecture as disclosed in FIG. 1. The embodiments disclosed herein further conducted experiments using a trained model based on the Resnet50 architecture. The Resnet50 from OpenAIs CLIPS library (i.e., a public library) was used to generate image features for 72105 training images and 10477 test images. A logistic regression was trained on the features and radiology reports of training images, then tested on features and labels of testing images. For each of the 39 labels an ROC-AUC was calculated. The average of all 39 ROC-AUC for the trained machine learning model is 0.7761819903717145. By comparison, the average of all 39 ROC-AUC for OpenAI Clip (i.e., a state-of-the-art method) is 0.7613616312748511. Table 4 illustrates a comparison of the ROC-AUC for each of the 39 labels between the trained machine learning model and OpenAI Clip. The comparisons show that the trained machine learning model improves the performance over the prior art.

TABLE 4
Comparison of ROC-AUC between a machine learning
model based on Resnet50 and OpenAI Clip.

| | Trained machine | |
|---|---|---|
| | learning model | OpenAI Clip |
| Cardiomegaly | 0.8579150361800206 | 0.799211345688456 |
| Left Atrial Enlargement | 0.8743810826915014 | 0.8354264780526558 |
| Left Ventricular Enlargement | 0.903284009572383 | 0.8351498121257405 |
| Right Atrial Enlargement | 0.8159503525733066 | 0.8074343511693947 |
| Right Ventricular Enlargement | 0.8151613705334184 | 0.7781402406288676 |
| Main Pulmonary Artery | 0.9433037277560247 | 0.8714544933626205 |
| Enlargement | | |
| Aortic Abnormality | 0.594625609639476 | 0.6181027063211246 |
| Heart Base Mass Effect | 0.8006092642126269 | 0.785006156328281 |
| Spondylosis | 0.8247831875983215 | 0.806173154243517 |
| Liver Abnormality | 0.8348684855950033 | 0.8278767299985101 |
| Ex. Thoracic or abdominal mass | 0.7254602058858624 | 0.7500077184251742 |
| Sign(s) of IVDD | 0.7881090658662828 | 0.7630785408357577 |
| Gastric Foreign Material | 0.6210308139224038 | 0.6307650906239418 |
| Cervical Tracheal Narrowing or | 0.9092590169000848 | 0.8959528412262416 |
| Opacity | | |
| Degenerative Joint Disease | 0.7246578098418054 | 0.7266930383097677 |
| Decreased serosal detail | 0.7268152460003054 | 0.7459913223920014 |
| Gastric Distention | 0.7236854163433327 | 0.7458058961340339 |
| Aggressive Bone Lesion | 0.6364975818243167 | 0.6285963333708244 |
| Fracture and/or Luxation | 0.6074535352398578 | 0.5597341536306912 |
| Esophagal Dilation | 0.7216417487824216 | 0.7304895443991393 |
| Intrathoracic Tracheal Narrowing | 0.926123077769525 | 0.8755784942959987 |
| Tracheal Deviation | 0.8741560630912736 | 0.8250140828735689 |
| Mediastinal Mass | 0.7673274842586377 | 0.7864508422028318 |
| Mediastinal Lymph Node | 0.6692351871350627 | 0.705762419833445 |
| Enlargement (any) | | |
| Sign(s) of Pleural Effusion | 0.875619925597103 | 0.8437935307792912 |
| Pneumothorax | 0.6447917093911926 | 0.6561974488331077 |
| Bronchial (inc. old dog and breed | 0.7767208974027519 | 0.7811077802536923 |
| related) | | |
| Interstitial Unstructured (inc. old | 0.8452052894635876 | 0.8183652280763607 |
| dog and breed related) | | |
| Pulmonary Alveolar | 0.8110713792443645 | 0.8039960428223155 |
| Pulmonary Interstitial - Nodule | 0.7750431874404015 | 0.7669218756478116 |
| (Under 1 cm) | | |
| Pulmonary Vascular | 0.7105313308784202 | 0.6608972112762064 |
| Pulmonary Mass (Over 1 cm) | 0.7446491752922165 | 0.715408679101278 |
| Splenomegaly | 0.8003138470095146 | 0.8027600659475994 |
| Microcardia | 0.7417685085245407 | 0.7671535345798081 |
| Mediastinal Widening | 0.8521869311104724 | 0.838042540960046 |
| Pleural Fissure Lines | 0.8757417431176976 | 0.8147541656266134 |
| Subcutaneous Nodule | 0.7510262529832936 | 0.841527446300716 |
| Subcutaneous Mass | 0.691553462352849 | 0.6102271638071505 |
| Fat Opacity Mass (e.g., lipoma) | 0.6885396054752045 | 0.6380551192346137 |

The embodiments disclosed herein investigated the effectiveness of multimodal training methods on the performance of computer-vision models for disease classification on feline and canine radiographs. The embodiments disclosed herein disclose the contrastive radiology captioning model, which utilizes a novel model architecture for training multi-image/text pairs using contrastive and captioning loss. The embodiments disclosed herein show that after multimodal alignment, performance on a multi-label image classification task is significantly better for several findings and otherwise comparable to the ensemble of deployed models in the current RapidRead system.

Interestingly, some of the more intractable findings in the current system had the most significant performance increases when trained using this architecture. For example, the average precision score for ‘ingesta in the stomach’ was 52% higher using the contrastive radiology captioning model compared to the current ensemble. This may be because the current system uses supervised training methods whereby the majority of labels are derived using an NLP algorithm. This algorithm may use rules to extract labels from radiology reports. This multimodal method of the contrastive radiology captioning model, however, can use the text itself as the ground-truth label. This may mean that it can capture nuances in the text that may be missed by an NLP labeler e.g., syntactic variability in how the pathology is described. Hence, this disclosure suggests that alignment between radiology reports and x-ray images may lead to better representation learning on pathology's that are difficult to label using alternative methods.

Ablation studies were also conducted to understand the importance of different parts of the architecture. This disclosure first shows that training a StudyFormer model using ImageNet weights leads to significantly worse results compared to when trained with the contrastive radiology captioning model trained weights. This demonstrates that performance improvements can be from multimodal training and not just a result of using the StudyFormer architecture itself. Then, this disclosure highlights the importance of the multimodal decoder by showing that its removal leads to a significant performance decrease when compared to the contrastive radiology captioning model trained StudyFormer.

In the embodiments disclosed herein, the maximum number of images used per study was 5.75% of studies have 5 images or less. Hence, 5 was chosen as it captures all the images in the majority of the studies whilst keeping the requirement for padded images and computational cost low. However, this can mean that 25% of studies had a surplus of unusable images. Hence, some of the images that were referenced in the study report were not available for the model to access. This may have impacted the ability of the model to accurately align images and reports in the embedding space.

The embodiments disclosed herein can have implications for the future development and deployment of deep learning models in the radiology domain. This disclosure demonstrates that multimodal methods can be used to train models using multiple x-ray images and their corresponding reports. This can allow for the utilization of large unlabeled data sets, without the need for alternative labelling methods such as NLP algorithms. Specifically, this disclosure shows that this method of training may be specifically beneficial in the radiology domain on findings that are difficult to reliably detect when using alternative labelling methods.

The embodiments disclosed herein also highlight the potential for the use of large language models for automating the radiology report writing process. This disclosure shows that by simply providing the contrastive radiology captioning model with unseen x-ray images, the model can generate text that closely resembles human diagnostic reports.

In conclusion, this disclosure shows that the architecture of the contrastive radiology captioning model can be a powerful method of training deep learning models when compared to supervised learning with alternative labelling methods. This disclosure also highlights the potential for automated diagnostic report generation by comparing actual reports to those generated by the contrastive radiology captioning model. Overall, the embodiments disclosed herein demonstrate the potential benefits of using the architecture of the contrastive radiology captioning model to train models for image classification tasks when using large, unlabeled datasets with multi-image/text pair inputs.

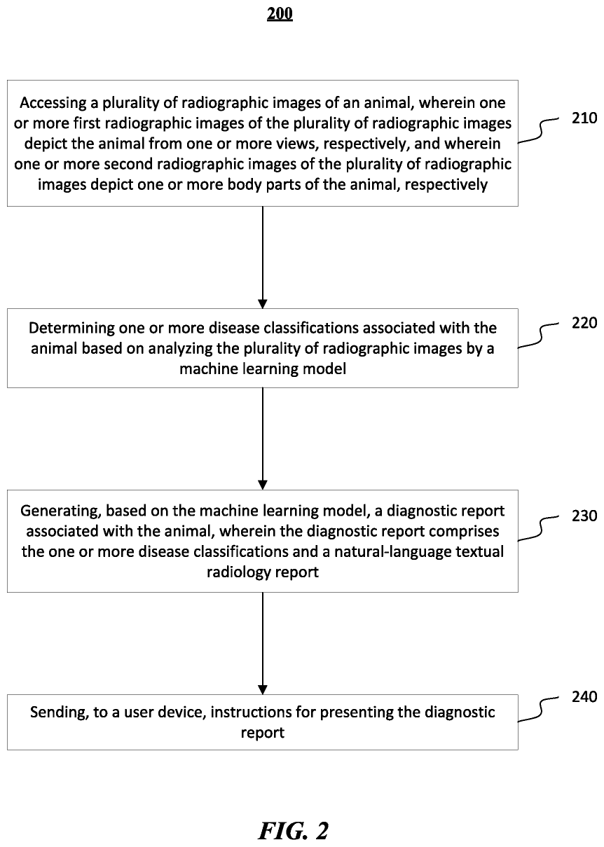

FIG. 2 illustrates an example method 200 for abnormality detection of radiographic images of animals. At step 210, one or more computing systems can access a plurality of radiographic images of an animal, wherein one or more first radiographic images of the plurality of radiographic images depict the animal from one or more views, respectively, and wherein one or more second radiographic images of the plurality of radiographic images depict one or more body parts of the animal, respectively. At step 220, the computing systems can determine one or more disease classifications associated with the animal based on analyzing the plurality of radiographic images by a machine learning model. At step 230, the computing systems can generate, based on the machine learning model, a diagnostic report associated with the animal, wherein the diagnostic report comprises the one or more disease classifications and a natural-language textual radiology report. At step 240, the computing systems can send, to a user device, instructions for presenting the diagnostic report.

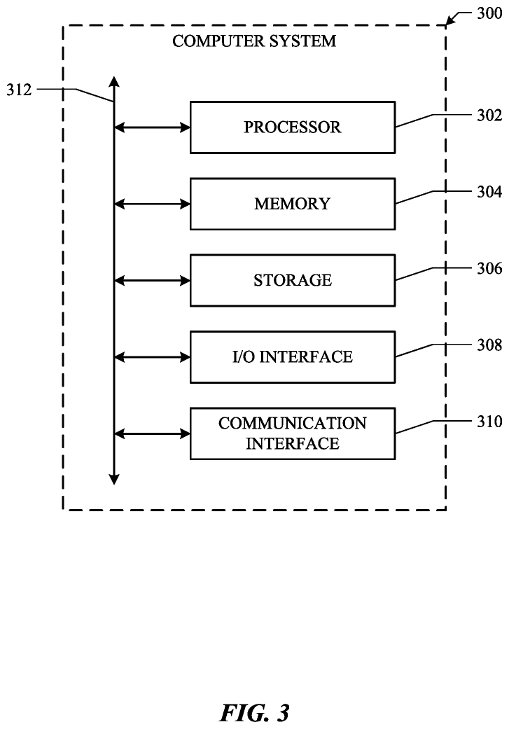

FIG. 3 illustrates an example computer system 300 or device used to facilitate abnormality detection from radiographic images of animals. In certain non-limiting embodiments, one or more computer systems 300 perform one or more steps of one or more methods described or illustrated herein. In certain other non-limiting embodiments, one or more computer systems 300 provide functionality described or illustrated herein. In certain non-limiting embodiments, software running on one or more computer systems 300 performs one or more steps of one or more methods described or illustrated herein or provides functionality described or illustrated herein. Some non-limiting embodiments include one or more portions of one or more computer systems 300. Herein, reference to a computer system can encompass a computing device, and vice versa, where appropriate. Moreover, reference to a computer system can encompass one or more computer systems, where appropriate.

This disclosure contemplates any suitable number of computer systems 300. This disclosure contemplates computer system 300 taking any suitable physical form. As example and not by way of limitation, computer system 300 can be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 300 can include one or more computer systems 300; be unitary or distributed; span multiple locations; span multiple machines; span multiple data centers; or reside in a cloud, which can include one or more cloud components in one or more networks. Where appropriate, one or more computer systems 300 can perform without substantial spatial or temporal limitation one or more steps of one or more methods described or illustrated herein. As an example and not by way of limitation, one or more computer systems 300 can perform in real time or in batch mode one or more steps of one or more methods described or illustrated herein. One or more computer systems 300 can perform at different times or at different locations one or more steps of one or more methods described or illustrated herein, where appropriate.

In certain non-limiting embodiments, computer system 300 includes a processor 302, memory 304, storage 306, an input/output (I/O) interface 308, a communication interface 410, and a bus 412. Although this disclosure describes and illustrates a particular computer system having a particular number of particular components in a particular arrangement, this disclosure contemplates any suitable computer system having any suitable number of any suitable components in any suitable arrangement.

In some non-limiting embodiments, processor 302 includes hardware for executing instructions, such as those making up a computer program. As an example and not by way of limitation, to execute instructions, processor 302 can retrieve (or fetch) the instructions from an internal register, an internal cache, memory 304, or storage 306; decode and execute them; and then write one or more results to an internal register, an internal cache, memory 304, or storage 306. In certain non-limiting embodiments, processor 302 can include one or more internal caches for data, instructions, or addresses. This disclosure contemplates processor 302 including any suitable number of any suitable internal caches, where appropriate. As an example and not by way of limitation, processor 302 can include one or more instruction caches, one or more data caches, and one or more translation lookaside buffers (TLBs). Instructions in the instruction caches can be copies of instructions in memory 304 or storage 306, and the instruction caches can speed up retrieval of those instructions by processor 302. Data in the data caches can be copies of data in memory 304 or storage 306 for instructions executing at processor 302 to operate on; the results of previous instructions executed at processor 302 for access by subsequent instructions executing at processor 302 or for writing to memory 304 or storage 306; or other suitable data. The data caches can speed up read or write operations by processor 302. The TLBs can speed up virtual-address translation for processor 302. In some non-limiting embodiments, processor 302 can include one or more internal registers for data, instructions, or addresses. This disclosure contemplates processor 302 including any suitable number of any suitable internal registers, where appropriate. Where appropriate, processor 302 can include one or more arithmetic logic units (ALUs); be a multi-core processor; or include one or more processors 302. Although this disclosure describes and illustrates a particular processor, this disclosure contemplates any suitable processor.

In some non-limiting embodiments, memory 304 includes main memory for storing instructions for processor 302 to execute or data for processor 302 to operate on. As an example and not by way of limitation, computer system 300 can load instructions from storage 306 or another source (such as, for example, another computer system 300) to memory 304. Processor 302 can then load the instructions from memory 304 to an internal register or internal cache. To execute the instructions, processor 302 can retrieve the instructions from the internal register or internal cache and decode them. During or after execution of the instructions, processor 302 can write one or more results (which can be intermediate or final results) to the internal register or internal cache. Processor 302 can then write one or more of those results to memory 304. In some non-limiting embodiments, processor 302 executes only instructions in one or more internal registers or internal caches or in memory 304 (as opposed to storage 306 or elsewhere) and operates only on data in one or more internal registers or internal caches or in memory 304 (as opposed to storage 306 or elsewhere). One or more memory buses (which can each include an address bus and a data bus) can couple processor 302 to memory 304. Bus 412 can include one or more memory buses, as described below. In certain non-limiting embodiments, one or more memory management units (MMUs) reside between processor 302 and memory 304 and facilitate accesses to memory 304 requested by processor 302. In certain other non-limiting embodiments, memory 304 includes random access memory (RAM). This RAM can be volatile memory, where appropriate. Where appropriate, this RAM can be dynamic RAM (DRAM) or static RAM (SRAM). Moreover, where appropriate, this RAM can be single-ported or multi-ported RAM. This disclosure contemplates any suitable RAM. Memory 304 can include one or more memories 304, where appropriate. Although this disclosure describes and illustrates a particular memory component, this disclosure contemplates any suitable memory.

In some non-limiting embodiments, storage 306 includes mass storage for data or instructions. As an example and not by way of limitation, storage 306 can include a hard disk drive (HDD), a floppy disk drive, flash memory, an optical disc, a magneto-optical disc, magnetic tape, or a Universal Serial Bus (USB) drive or a combination of two or more of these. Storage 306 can include removable or non-removable (or fixed) media, where appropriate. Storage 306 can be internal or external to computer system 300, where appropriate. In certain non-limiting embodiments, storage 306 is non-volatile, solid-state memory. In some non-limiting embodiments, storage 306 includes read-only memory (ROM). Where appropriate, this ROM can be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 306 taking any suitable physical form. Storage 306 can include one or more storage control units facilitating communication between processor 302 and storage 306, where appropriate. Where appropriate, storage 306 can include one or more storages 306. Although this disclosure describes and illustrates particular storage, this disclosure contemplates any suitable storage.

In certain non-limiting embodiments, I/O interface 308 includes hardware, software, or both, providing one or more interfaces for communication between computer system 300 and one or more I/O devices. Computer system 300 can include one or more of these I/O devices, where appropriate. One or more of these I/O devices can enable communication between a person and computer system 300. As an example and not by way of limitation, an I/O device can include a keyboard, keypad, microphone, monitor, mouse, printer, scanner, speaker, still camera, stylus, tablet, touch screen, trackball, video camera, another suitable I/O device or a combination of two or more of these. An I/O device can include one or more sensors. This disclosure contemplates any suitable I/O devices and any suitable I/O interfaces 308 for them. Where appropriate, I/O interface 308 can include one or more device or software drivers enabling processor 302 to drive one or more of these I/O devices. I/O interface 308 can include one or more I/O interfaces 308, where appropriate. Although this disclosure describes and illustrates a particular I/O interface, this disclosure contemplates any suitable I/O interface.

In some non-limiting embodiments, communication interface 410 includes hardware, software, or both providing one or more interfaces for communication (such as, for example, packet-based communication) between computer system 300 and one or more other computer systems 300 or one or more networks. As an example and not by way of limitation, communication interface 410 can include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 410 for it. As an example and not by way of limitation, computer system 300 can communicate with an ad hoc network, a personal area network (PAN), a local area network (LAN), a wide area network (WAN), a metropolitan area network (MAN), or one or more portions of the Internet or a combination of two or more of these. One or more portions of one or more of these networks can be wired or wireless. As an example, computer system 300 can communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 300 can include any suitable communication interface 410 for any of these networks, where appropriate. Communication interface 410 can include one or more communication interfaces 410, where appropriate. Although this disclosure describes and illustrates a particular communication interface, this disclosure contemplates any suitable communication interface.

In certain non-limiting embodiments, bus 412 includes hardware, software, or both coupling components of computer system 300 to each other. As an example and not by way of limitation, bus 412 can include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 412 can include one or more buses 412, where appropriate. Although this disclosure describes and illustrates a particular bus, this disclosure contemplates any suitable bus or interconnect.

Herein, a computer-readable non-transitory storage medium or media can include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium can be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments can include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates some non-limiting embodiments as providing particular advantages, certain non-limiting embodiments can provide none, some, or all of these advantages.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications can be made to the elements and operations described above to obtain a result that remains within the scope of the systems and processes described in this disclosure.

The embodiments disclosed herein are only examples, and the scope of this disclosure is not limited to them. Certain non-limiting embodiments can include all, some, or none of the components, elements, features, functions, operations, or steps of the embodiments disclosed above. Embodiments are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

Various references are cited in this document, which are hereby incorporated by reference in their entireties herein.

1. A method comprising, by one or more computing systems:
accessing a plurality of radiographic images of an animal, wherein one or more first radiographic images of the plurality of radiographic images depict the animal from one or more views, respectively, and wherein one or more second radiographic images of the plurality of radiographic images depict one or more body parts of the animal, respectively;
determining one or more disease classifications associated with the animal based on analyzing the plurality of radiographic images by a machine learning model;
generating, based on the machine learning model, a diagnostic report associated with the animal, wherein the diagnostic report comprises the one or more disease classifications and a natural-language textual radiology report; and
sending, to a user device, instructions for presenting the diagnostic report.

2. The method of claim 1, wherein each of the plurality of radiographic images is formatted as a Digital Imaging and Communications in Medicine (“DICOM”) image.

3. The method of claim 1, wherein the machine learning model is based on at least one first neural network and at least one second neural network, the at least one first neural network and the at least one second neural network being coupled with each other.

4. The method of claim 1, wherein generating the diagnostic report comprises:
accessing a plurality of reference reports;
encoding the plurality of reference reports into a feature space;
encoding the plurality of radiographic images into the feature space; and
determining the diagnostic report based on similarity search in the feature space.

5. The method of claim 1, wherein one of the one or more disease classifications indicates an abnormal tissue.

6. The method of claim 5, further comprising:
identifying the abnormal tissue as at least one of cardiovascular, pulmonary structure, mediastinal structure, pleural space, or extra thoracic.

7. The method of claim 1, further comprising:
accessing a plurality of training radiographic images, wherein the plurality of training radiographic images are associated with a plurality of training radiology reports, respectively; and
training the machine learning model based on the accessed training radiograph images and their respective training radiology reports.

8. The method of claim 7, further comprising:
preprocessing each of plurality of training radiographic images, wherein the preprocessing comprises one or more of padding, random augmentation, random flip, Gaussian blur, or normalization.

9. The method of claim 7, further comprising:
applying long document encoding to each of the plurality of training radiology reports.

10. The method of claim 7, further comprising:
preprocessing each of plurality of training radiology reports, wherein the preprocessing comprises one or more of tokenization, padding, adding a classification token, or applying an attention mask.

11. The method of claim 1, wherein the machine learning model comprises an image encoder, a multi-image encoder, a text decoder, and a multimodal decoder.

12. The method of claim 11, further comprising:
generating, by the image encoder, a feature map based on the plurality of radiologic images;
generating, by the multi-image encoder based on the feature map, one or more multi-image keys and values; and
generating, by the multimodal decoder based on the one or more multi-image keys and values and a start of sentence token, the natural-language textual radiology report.

13. The method of claim 1, wherein the diagnostic report further comprises one or more of the plurality of radiologic images.

14. One or more computer-readable non-transitory storage media embodying software that is operable when executed to:
access a plurality of radiographic images of an animal, wherein one or more first radiographic images of the plurality of radiographic images depict the animal from one or more views, respectively, and wherein one or more second radiographic images of the plurality of radiographic images depict one or more body parts of the animal, respectively;
determine one or more disease classifications associated with the animal based on analyzing the plurality of radiographic images by a machine learning model;
generate, based on the machine learning model, a diagnostic report associated with the animal, wherein the diagnostic report comprises the one or more disease classifications and a natural-language textual radiology report; and
send, to a user device, instructions for presenting the diagnostic report.

15. (canceled)

16. The media of claim 14, wherein the machine learning model is based on at least one first neural network and at least one second neural network, the at least one first neural network and the at least one second neural network being coupled with each other.

17.-23. (canceled)

24. The media of claim 14, wherein the machine learning model comprises an image encoder, a multi-image encoder, a text decoder, and a multimodal decoder.

25. The media of claim 24, wherein the software is further operable when executed to:
generate, by the image encoder, a feature map based on the plurality of radiologic images;
generate, by the multi-image encoder based on the feature map, one or more multi-image keys and values; and
generate, by the multimodal decoder based on the one or more multi-image keys and values and a start of sentence token, the natural-language textual radiology report.

26. (canceled)

27. A system comprising: one or more processors; and a non-transitory memory coupled to the processors comprising instructions executable by the processors, the processors operable when executing the instructions to:
access a plurality of radiographic images of an animal, wherein one or more first radiographic images of the plurality of radiographic images depict the animal from one or more views, respectively, and wherein one or more second radiographic images of the plurality of radiographic images depict one or more body parts of the animal, respectively;
determine one or more disease classifications associated with the animal based on analyzing the plurality of radiographic images by a machine learning model;
generate, based on the machine learning model, a diagnostic report associated with the animal, wherein the diagnostic report comprises the one or more disease classifications and a natural-language textual radiology report; and
send, to a user device, instructions for presenting the diagnostic report.

28.-36. (canceled)

37. The system of claim 27, wherein the machine learning model comprises an image encoder, a multi-image encoder, a text decoder, and a multimodal decoder.

38. The system of claim 37, wherein the processors are further operable when executing the instructions to:
generate, by the image encoder, a feature map based on the plurality of radiologic images;
generate, by the multi-image encoder based on the feature map, one or more multi-image keys and values; and
generate, by the multimodal decoder based on the one or more multi-image keys and values and a start of sentence token, the natural-language textual radiology report.

39. (canceled)
